(19) United States
(12) Patent Application Publication
(10) Pub. No.: US 2017/0106148 A1
(43) Pub. Date: Apr. 20, 2017

(54) Injection Stick

(71) Applicant: Daniel Nagase, Vancouver (CA)

(72) Inventor: Daniel Nagase, Vancouver (CA)

(21) Appl. No.: 14/886,074

(22) Filed: Oct. 18, 2015

(51) Int. Cl. A61M 5/31; A61M 5/32

(52) U.S. Cl. CPC A61M 5/31; A61M 5/3202; A61M 2205/02

(57) ABSTRACT

The injection stick is a pole like drug delivery device that allows administration of injection medications from a distance. The design allows for an operator to maintain a safe distance away from a dangerous subject receiving injections. Unlike other injection systems, the injection stick features two design elements to enhance safety and prevent the device from being turned against it's operator by an assailant. The first is a continuously sliding mesh of nylon or other fabric that surrounds all parts of the device except the handle, which simply slides from the outside of the shaft to the inside of the shaft when the device is grabbed and pulled by anyone other than the operator. The second feature is variable flexibility of the shaft, which can be altered from flexible and bendable to stiff depending on the tension applied to the sliding fabric surrounding the shaft of the device.

### CROSS-REFERENCE TO RELATED APPLICATIONS

U.S. Pat. No. 4,684,366 Denny , et al. Aug. 4, 1987 Syringe for the remote injection of animals and fish
U.S. Pat. No. 5,202,533 Vandersteen Apr. 13, 1993 Drug injection apparatus for an animal

U.S. Pat. No. 5,531,685 Hemmer, et al. Jul. 2, 1996 Steerable variable stiffness device

### STATEMENT REGARDING FEDERALLY SPONSORED RESEARCH OR DEVELOPMENT

(if Applicable)

Not Applicable

### REFERENCE TO SEQUENCE LISTING, A TABLE, OR A COMPUTER PROGRAM LISTING COMPACT DISC APPENDIX (IF APPLICABLE)

Not Applicable

### BACKGROUND OF THE INVENTION

Urban emergency departments worldwide encounter situations of psychotic or violent patients on a daily basis. In order to treat patients for drug overdoses or medical illnesses that may underlie violent or dangerous behavior, injection antipsychotics and sedatives are sometimes required—especially in situations where verbal attempts to ellicit cooperation have failed. As violent patients are often unwilling to take medications orally, injections are often a necessity. However, administering an injection in a violent situation comes at great risk to both the health care provider and patient. Often multiple personnel need to be present to physically subdue the patient first before an injection is given. The device described below facilitates the safe administration of a hypodermic dose of sedative in a manner that is safe for both the patient and caregiver via a flexible pole long enough to put the caregiver out of striking range of the subject.

The benefits of administering a hypodermic injection of sedative via an extended pole are two-fold. First, it enhances safety for the healthcare provider by providing distance between the provider and patient. Secondly, it prevents transmission of infectious diseases from a patient to a caregiver. However, any pole like device in a hostile situation, can be turned against its user. In order to prevent such a happening, this patent covers the implementation of the following design paradigms to protect the operator of the device.

First is a mechanism to prevent the injection stick from being grabbed and taken away from the user by the subject or assailant. Second is a mechanism to change the rigidity of the pole from flexible and harmless, to rigid for accurate positioning and administration of injection, then back to flexible again in order to minimize harm. This mechanism for rigidity adjustment must be fast enough that the pole can be made rigid immediately before application of the sedative injection, with a user triggered return to flexibility immediately after injection.

### Rationale:

Whereas injection sedatives can be very beneficial in providing healthcare in dangerous situations, the close proximity required for administration of an injection presents a safety risk to the healthcare provider. An extended pole capable of providing a hypodermic injection, but from a distance, mitigates this risk. A flexible pole composed of a system of sliding nylon netting or fabric over a hollow flexible delivery shaft stymies an assailants grip on the pole causing the shaft of the device to “slip out” of the assailant's grip whenever grabbed at any part except the handle. This mitigates the risk of a rigid remote delivery injection system being turned against the operator as a weapon. Implementation of a rigidity adjustment mechanism further decreases the risk of harm by making the device flexible when not being actively positioned and injecting. This is done by varying the tension applied to the fabric or netting that surrounds the pole over both the inner and outter surfaces of the hollow flexible shaft. The hollow flexible shaft itself is comprised of a number of low friction, longitudinally rigid, but laterally flexible elements as described in the patent.

### BRIEF SUMMARY OF THE INVENTION

The injection stick drug delivery system is comprised of 3 functional parts. The first part is a hollow shaft comprised of a cylindrical formation of flexible poles held in a circle by two retainer rings at the proximal and distal ends of the shaft. This shaft is enveloped on both the outer and inner surfaces by nylon netting or other low friction fabric that is free to slide both circumferentially around the shaft, and longitudinally along the shaft. As the fabric slides longitudinally along the outer surface of the shaft, it wraps around the end of the shaft and passes through to the inner surface of the hollow shaft. Simultaneously, the fabric or nylon netting from the inside of the shaft wraps around the opposite end of the shaft to take the place of the fabric that has been moved from outside of the hollow shaft. This provides a continuous and ungrippable surface to the outside of the hollow shaft.

The second functional component is a handle at the operator end of the shaft that allows the shaft to be moved and directed in space. Integral in the handle assembly is a friction mechanism that can first stop the nylon netting or fabric from sliding from the outside to the inside of the hollow shaft, and then secondly apply tension to the remaining nylon netting to make rigid the assembly of flexible poles comprising the hollow shaft via tension applied to the hollow shaft through the nylon netting or fabric.

The third and last functional component is a syringe and carrier mechanism that passes through the center of the hollow shaft to the distal end of the device. At the distal end of the device the syringe is held by a carrier such that the tip of the needle does not extend past the tip of the device. Prior to delivering a measured dose of medication via a hypodermic needle, the syringe needle is mechanically or electrically actuated to extend out from the end of the hollow shaft. If actuation is mechanical, it is performed in two parts via flexible shafts placed behind the syringe and syringe carrier assembly. The first step in actuation is a hollow flexible pole to push the syringe forward in the carrier assembly to extend the needle tip out of the opening at the end of the injection stick device. The second step proceeds via a second flexible pole that pushes the plunger of the syringe to deliver the medication through the end of the needle. One or both steps in actuation can be alternatively performed electrically via servo motors instead of mechanically.

### BRIEF DESCRIPTION OF THE SEVERAL VIEWS OF THE DRAWING

FIG. 1: The functionally unique parts of the injection stick are shown without the handle and covering mesh or fabric.
i) The retainer ring covers are shown bounding the longitudinal elements that form the shaft of the device.
ii) Only the functional part of the outer shell is shown, without the handle assembly that forms the rest of the outer shell.
iii) The friction funnel is the functional component of the inner shell assembly, and only that is shown. The rest of the inner shell assembly is integrated into the handle of the outer shell.

The syringe and carrier assembly, hollow pushrod, plunger pushrod, and trigger assembly for actuation of the hollow pushrod and plunger pushrods are not shown.

FIG. 2: Same as figure one, but a perpendicular view showing the front of the device that points towards the subject receiving the injection.

FIG. 3: A view of the overall form taken by the continuous loop of fabric or mesh that covers the shaft and retainer ring covers of the device.

FIG. 4: A semi transparent view of the device with the mesh or fabric covering the shaft and retainer ring covers.

FIG. 5: A perpendicular view of FIG. 4 showing the back (or operator) end of the device.

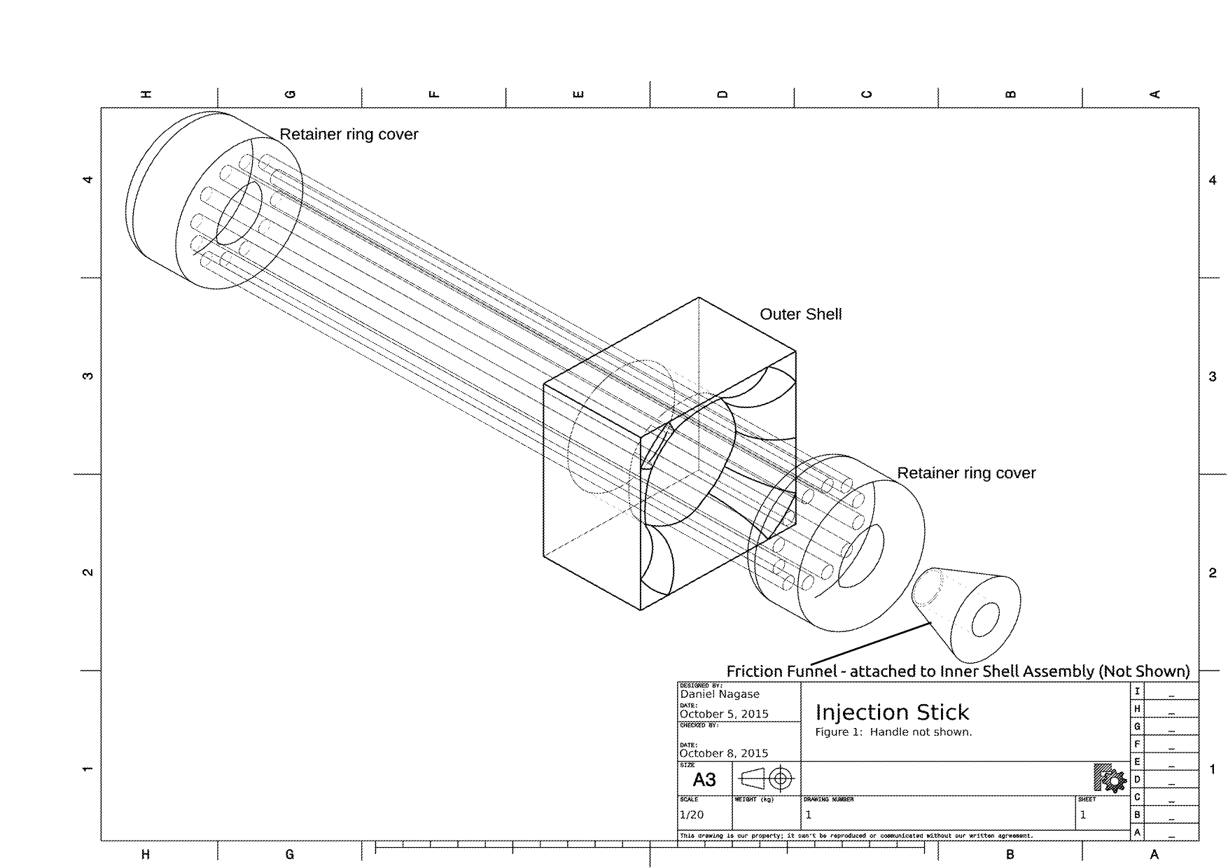

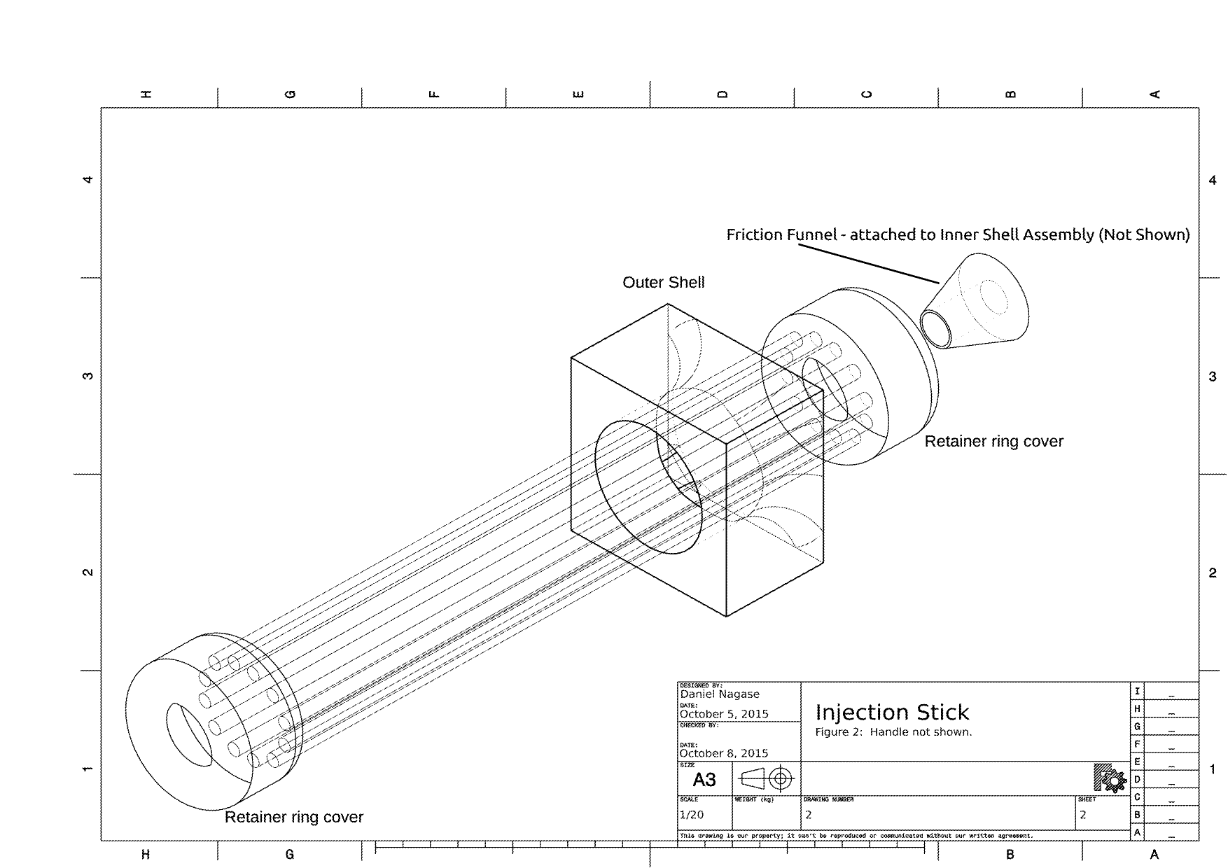

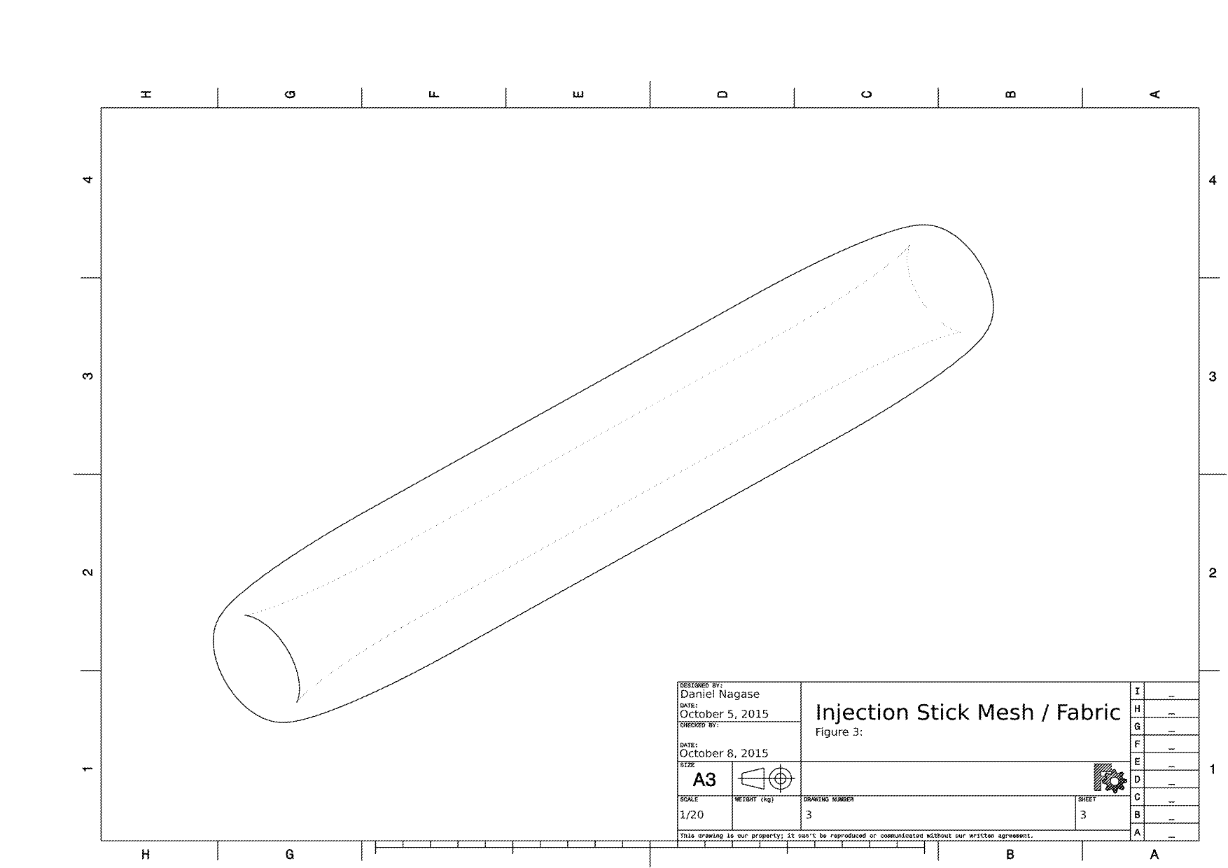

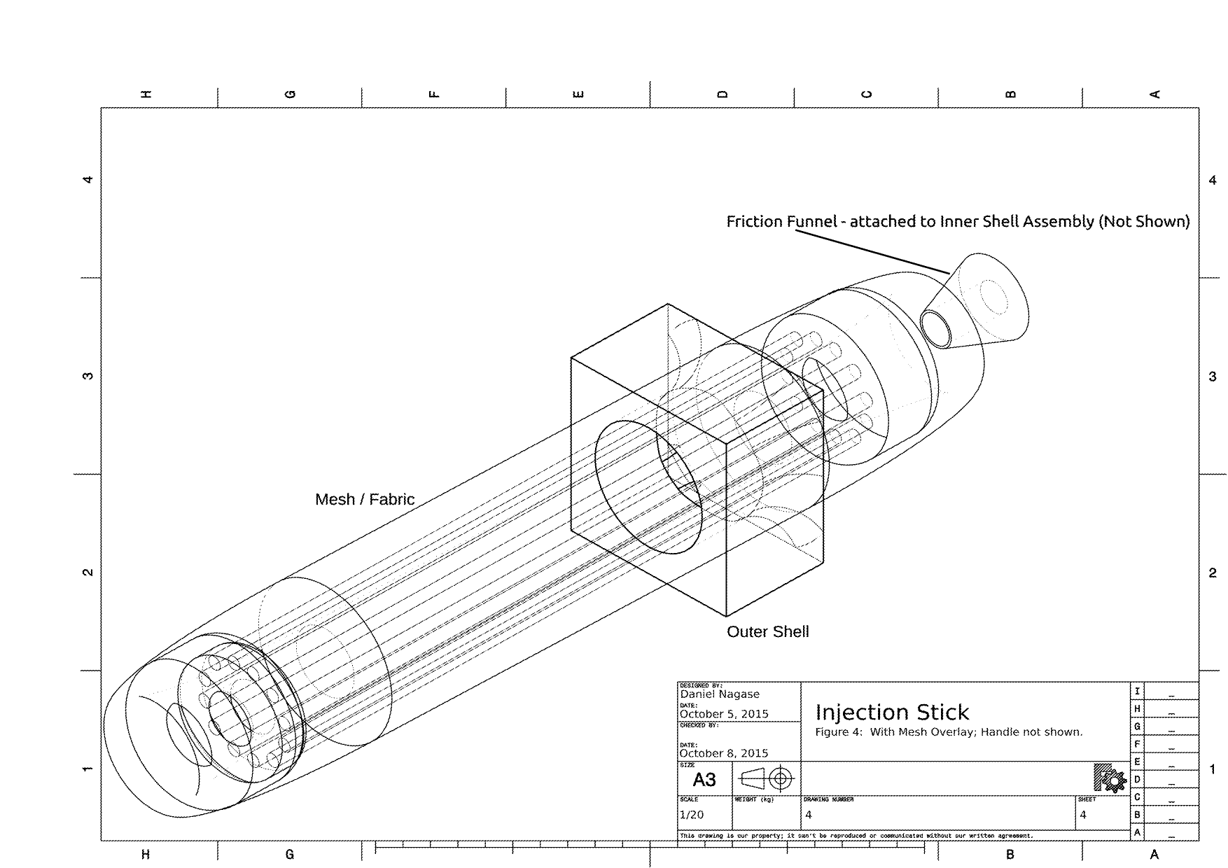

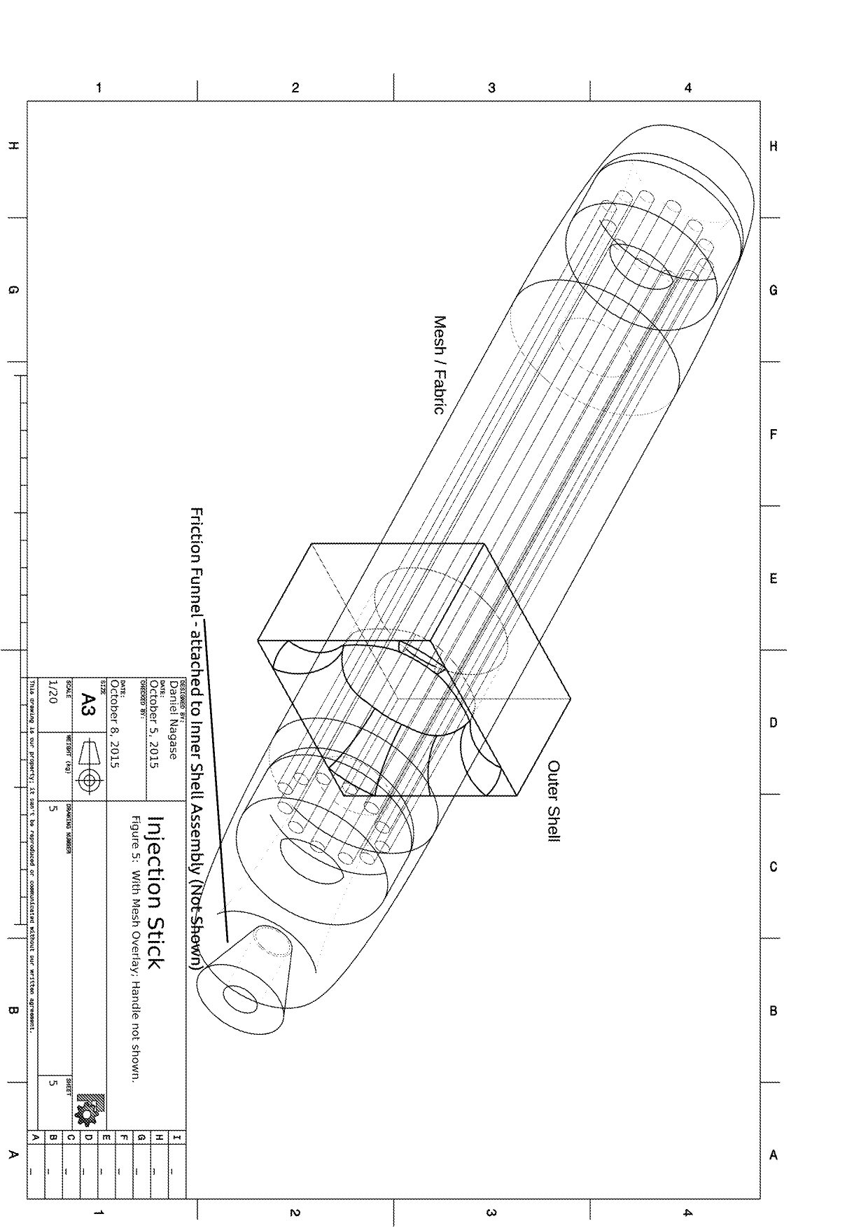

### DETAILED DESCRIPTION OF THE INVENTION

The “injection stick” extended pole injection system consists of 8 components:
1) A shaft composed of a circumferential assembly of longitudinal structural elements:
   a) Each longitudinal element has the following property, longitudinal rigidity with lateral and torsional flexibility.
   b) A number of these longitudinal elements are arranged in a circle to form a long tube bounded by retainer rings on either end that hold each of the longitudinal elements together in a circumferential manner.
   c) Each longitudinal element may be constructed of fibreglass, carbon fibre or other flexible material shaped such that it offers lateral flexibility and longitudinal rigidity.
2) Retainer Rings with integrated low friction cover:
   a) Retainer rings at either end of the injection device shaft hold each longitudinal structural element in a circular formation.
   b) Each retainer ring is held onto each longitudinal structural element via metal crimps or bonded stoppers locked around each longitudinal element on either side of the retainer ring. The spacing of each crimp or bump stop on the longitudinal structural element allows for small movements of each longitudinal element forward and backwards through each retainer ring. The distance between the crimps allow for a measure of longitudinal play for each longitudinal element within each retainer ring.
   b) Retainer Ring Covers: These may either be separate detachable units or integrated with the retainer rings as one molded unit. The retainer ring covers serve to provide a smooth low friction surface over which nylon netting or other low friction fabric can slide over each end of the injection device shaft without snagging.
   c) The distal (injection end) retainer ring cover has an opening large enough to allow passage of a needle, but small enough to prevent a carrier+syringe assembly from passing through. The proximal retainer ring cover has an opening large enough to allow passage of a carrier+syringe assembly. This allows a syringe to be loaded into the shaft of the injection stick through the proximal retainer ring cover.
3) Nylon mesh or other Low Friction Fabric:
   a) A single contiguous piece of fabric, or net made of high tensile nylon—similar to fishing netting, that wraps around the shaft formed by the longitudinal elements. The single mesh covers both the inner and outer surfaces of the long cylindrical tube formed by the longitudinal structural elements and retainer rings.
   b) This mesh or fabric can slide freely over the low friction covers around each retainer ring. Grabbing and pulling the mesh surrounding the outside of the tube will only result in the mesh on the outside of the tube sliding over the longitudinal structural elements, around the retainer ring covers, and over into the inner surface of the tube formed by the longitudinal structural elements. Simultaneously, the mesh on the inside of the tube is fed out of the opposite retainer ring cover, to wrap around and over to the outside of the tube formed by the longitudinal structural assembly.
4) Syringe and Carrier assembly:
   a) A carrier shuttle holds a standard hypodermic syringe such that it can be loaded into the hollow shaft assembly described in parts #1 and #2 above.
   b) The syringe +carrier are threaded through to the end of the hollow shaft assembly until the carrier abuts the inner opening of the distal retainer ring cover.
   c) The carrier assembly holds the syringe such that the needle of the syringe is not exposed past the end of the distal retainer ring cover.
   d) The carrier has a spring assembly that allows the syringe to be advanced forward within the carrier to extend the needle of the syringe past the end of the distal retainer ring cover, prior to injection.
5) Flexible Hollow Pushrod:
   a) The carrier assembly is advanced within the tube formed by the longitudinal elements via a flexible hollow pushrod until it abuts the inner opening of the distal retainer ring cover.
   b) The syringe is held within the carrier such that the needle is not exposed past the end of the distal retainer ring cover until a force is applied by the hollow flexible pushrod to force the syringe forward in the carrier. This extends the tip of the needle attached to the end of the syringe, past the end of the distal retainer ring cover, allowing the needle penetrate the skin of the subject.
   c) When the force upon the pushrod is removed, the syringe body retracts back within the carrier, withdrawing the previously exposed needle tip back within the bounds of the distal retainer ring cover.
6) Flexible Plunger Pushrod:
   a) A flexible plunger pushrod, pushes the plunger of the syringe to administer the drug through the needle of the syringe.
   b) The flexible plunger pushrod pushes on the plunger of the syringe independently of the Flexible Hollow Pushrod—which only pushes the syringe forward in the carrier.
7) Handle assembly:
   a) The handle assembly consists of three functional elements integrated into an ergonomic handle grip.
      1. Outer Shell
      2. Inner shell
      3. Friction funnel p2 b) The outer shell clamps over the proximal retainer ring cover such that the retainer ring cover and longitudinal element assembly cannot be pulled away from the handle. The outer shell is the main functional component of the handle assembly.
   c) The outer shell holds the proximal retainer ring cover snugly such that the outer shell can move the whole cylindrical longitudinal structural element assembly via physical contact with the proximal retainer ring cover through the fabric or mesh overlying the proximal retainer ring cover.
   d) The contact portion of the outer shell with the mesh or fabric surrounding the proximal retainer ring cover is a friction variable surface. At low to moderate pressures, the contact surface of the outer shell offers low friction to the sliding mesh or fabric. At higher pressures it offers high friction to the nylon mesh or fabric.
   e) The functional part of the inner shell assembly is a truncated funnel shaped friction funnel attached to an inner shell. This inner shell is also integrated into the handle assembly. The funnel is made of a stiff high friction material such as rubber coated metal or plastic. The distal end of the truncated funnel passes partially into the central opening of the proximal retainer ring cover.
   f) An opening in the center of the friction funnel allows passage of the Flexible Hollow Pushrod and Flexible Plunger Pushrod through the center of the funnel.
   g) The inner shell is separated from, but attached to the outer shell and handle assembly by springs that serve to separate the two shells. Squeezing the handle assembly results in the two shells being squeezed together. This pushes the friction funnel part of the inner shell assembly forward against the inner surface of the proximal retainer ring cover. This applies friction to the nylon mesh or fabric passing through the inner side of the proximal retainer ring cover. This locks in place the fabric or mesh between the friction funnel and inner surface of the proximal retainer ring cover.
   h) Simultaneously, whilst the fabric or mesh is locked in place by the friction funnel, the proximal retainer ring cover is pushed forward into the outer shell by the friction funnel. The low to moderate friction contact portion of the outer shell that contacts the outer surface of the proximal retainer ring cover pulls tight the fabric or mesh running along the outside of the proximal retainer ring cover. This tension is possible because the previously mobile mesh or fabric is fixed in place between the friction plunger and the inner surface of the retainer ring cover, allowing the low to moderate friction surfaces of the outer shell to pull tight the nylon mesh running along the outside of the proximal retainer ring cover.
   i) When the nylon mesh or fabric is pulled taught, the circular assembly of longitudinal elements will transform into a stiff and inflexible assembly via the tension applied by the nylon mesh or low friction fabric. The injection stick, once stiff, allows for the accurate positioning and administration of medication through the syringe.
8) Trigger Assembly:
   a) Pushing the first trigger forces the syringe forward in the carrier assembly via the Flexible Hollow Pushrod, to extend the needle of the syringe out past the end of the injection stick.
   b) Pushing the second trigger pushes the Flexible Plunger Pushrod which pushes the plunger of the syringe to administer the medication through the extended needle.
   c) The first and second triggers can be assembled together with an intervening spring assembly such that both triggers are integrated into one. With light pressure on the integrated trigger, the syringe is pushed forward into the carrier by the Flexible Hollow Pushrod. This exposes the needle past the end of the distal retainer ring cover. Subsequently with firmer pressure on the integrated trigger, the Flexible Plunger Pushrod is actuated allowing the delivery of medication. In this way needle extension and medication delivery can be performed by the operator in a single motion via a single integrated two-step trigger.

1) A hollow shaft composed of flexible longitudinal elements arranged circumferentially, surrounded on both the inner and outer surfaces by a single continuous net of nylon or other low friction fabric.
i) When an attempt is made to grasp the shaft, the net or fabric simply slides around the shaft, stymieing any attempt to grab it.

2) A system to variably apply friction and tension to the mesh or fabric surrounding the hollow shaft described above.
i) Applying tension to the fabric surrounding the hollow shaft stiffens the above described circumferential assembly of flexible elements such that the hollow shaft formed by the circumferential assembly of longitudinal elements becomes rigid.

3) A system for drug delivery through the hollow shaft described in claims #1 and #2 via a standard hypodermic syringe held by a carrier passed through the lumen of the hollow shaft described in claim #1 and claim #2.

4) A spring loaded carrier holding a standard hypodermic syringe, such that the tip of the needle stays protected within the shaft described in claims #1 and #2, where pressure on the flange of the syringe from a hollow flexible pushrod variably advances the syringe within the spring loaded carrier to extend the tip of the hypodermic needle out from the end of the shaft described in claims #1 and #2.
i) Release of pressure on the flange of the syringe allows retraction of the syringe within the carrier to retract the needle back into the hollow shaft described in claim #1 and claim #2 after delivery of medication.

5) A single step operator actuated mechanism to both apply tension to the mechanism in claim #2 and extend the hypodermic needle forward in the carrier described in claim #4 such that the hypodermic needle extends past the distal end of the hollow shaft described in claim #1.

6) A second operator actuated mechanism, independent of the mechanism in claim #5, that can variably push the plunger of the syringe to administer a variable dose of medication.
